(19) United States
(12) Patent Application Publication
(10) Pub. No.: US 2024/0000408 A1
(43) Pub. Date: Jan. 4, 2024

(54) C-ARM IMAGING SYSTEM

(71) Applicant: GE Precision Healthcare LLC, Waukesha, WI (US)

(72) Inventors: Changliang Cai, Beijing (CN); Zhan Wang, Beijing (CN); Tiean Lei, Beijing (CN); Jundong Li, Beijing (CN); Da Sheng, Beijing (CN); Zifeng Xiao, Beijing (CN); Chao Jiang, Beijing (CN)

(21) Appl. No.: 18/345,999

(22) Filed: Jun. 30, 2023

(30) Foreign Application Priority Data
Jun. 30, 2022 (CN) 202210756230.5

(51) Int. Cl. A61B 6/00

(52) U.S. Cl. CPC A61B 6/4441; A61B 6/4452

(57) ABSTRACT

A C-arm imaging system includes a C-arm, and a sliding support assembly. The C-arm includes a first end portion and a second end portion which are oppositely disposed. The first end portion is used to connect to the X-ray tube assembly, while the second end portion is used to connect to the detector assembly, and the X-ray tube assembly and the detector assembly are aligned. The sliding support assembly is connected to the C-arm, and the C-arm can slide relative to the sliding support assembly, wherein the C-arm rotates at a rotational angle of not less than 90 degrees relative to the sliding support assembly from a first position to the first end portion or the second end portion, the first position being a position where the line connecting the center of the detector assembly and the center of the X-ray tube assembly is in a vertical direction.

### CROSS REFERENCE TO RELATED APPLICATIONS

This application claims priority to Chinese Patent Application No. 202210756230.5, filed on Jun. 30, 2022, the disclosure of which is incorporated herein by reference in its entirety.

### BACKGROUND

The present disclosure relates to medical imaging technology, and in particular to a C-arm imaging system.

Medical imaging systems can be used in various applications, including medical applications and industrial applications. In a medical environment, X-ray imaging systems can perform tissue and bone imaging for patients in a non-invasive manner. The X-ray imaging systems are capable of capturing a plurality of images at a specified interval, and sequentially displaying the images to create a single image of an object under test.

The X-ray imaging systems generate the image of the object under test by exposing the object to an energy source, for example, X-rays passing through the object. The generated image may be used for a variety of purposes. The X-ray imaging systems include suspended X-ray imaging systems, mobile X-ray imaging systems, C-arm imaging systems, and the like. The “C-arm” generally refers to an X-ray imaging device having a rigid and/or articulating structural member. The rigid and/or articulating structural member has an X-ray source and a detector assembly. The X-ray source and the detector assembly are respectively located at opposite end portions of the structural member, so that the X-ray source and the detector are disposed opposite to each other. The structural member is usually “C” shaped and is thus referred to as a C-arm. In this way, X-rays emitted by the X-ray source can strike the detector and provide an X-ray image of one or more objects placed between the X-ray source and the detector.

Generally, a C-arm imaging system includes a C-arm and a sliding support assembly slidingly attached to the C-arm. The C-arm can slide relative to the sliding support assembly, and such sliding includes sliding from a position where the detector is located directly above the X-ray tube (initial position) along the clockwise and counterclockwise direction. However, due to reasons such as that the X-ray tube is mounted at the end portion of the C-arm and so on, the C-arm cannot move to an upright “C” position, i.e., cannot move along the initial position to a position where the detector is located directly at the left side of the tube, which results in a limited rotation angle of the C-arm, and thus cannot achieve positioning and shooting at certain specific positions or angles.

### SUMMARY

This summary introduces concepts that are described in more detail in the detailed description. It should not be used to identify essential features of the claimed subject matter, nor to limit the scope of the claimed subject matter.

In an aspect of the disclosure, a C-arm imaging system, the imaging system comprising a C-arm and a sliding support assembly. The C-arm comprises a first end portion and a second end portion which are oppositely disposed. The first end portion is used to connect to the X-ray tube assembly, while the second end portion is used to connect to the detector assembly, and the X-ray tube assembly and the detector assembly are aligned. The sliding support assembly is connected to the C-arm, and the C-arm can slide relative to the sliding support assembly, wherein the C-arm slides at an angle not less than 90 degrees relative to the sliding support assembly from a first position to the first end portion or the second end portion, the first position being a position where the line connecting the center of the detector assembly and the center of the X-ray tube assembly is in a vertical direction.

In another aspect of the disclosure, a C-arm imaging system, the imaging system comprising a C-arm, an X-ray tube assembly, a detector assembly, and a sliding support assembly. The C-arm has a recess provided on an outer surface thereof, and the recess forms a sliding track of the C-arm. The X-ray tube assembly is mounted at the first end portion of the C-arm. The detector assembly is mounted at the second end portion of the C-arm. The sliding support assembly is connected to the outer surface of the C-arm, and the sliding support assembly comprises a sliding portion, at least a portion of the sliding portion being provided within the sliding track, wherein the sliding portion is at an angle not less than 90 degrees from a first position to the first end portion or second end portion, and the first position is a position where the line connecting the center of the detector assembly and the center of the X-ray tube assembly is in a vertical direction.

### BRIEF DESCRIPTION OF THE DRAWINGS

Various aspects of this disclosure may be better understood upon reading the detailed description and with reference to the attached drawings.

FIG. 1 is a schematic diagram of a prior art C-arm imaging system to illustrate limitations of the prior art design.

FIG. 2 is a schematic diagram showing a C-arm imaging system at a first position according to at least one embodiment of the present disclosure.

FIG. 3 is a schematic diagram of an X-ray tube assembly of the C-arm imaging system shown in FIG. 2 according to at least one embodiment of the present disclosure.

FIG. 4 is a schematic diagram of the C-arm shown in FIG. 2 according to at least one embodiment of the present disclosure.

FIG. 5 is a schematic diagram showing the C-arm imaging system at a second position according to at least one embodiment of the present disclosure.

FIG. 6 is a schematic diagram showing the C-arm imaging system at a third position according to at least one embodiment of the present disclosure.

FIG. 7 is a schematic diagram of a sliding support assembly of a C-arm imaging system according to at least one embodiment of the present disclosure.

FIG. 8 is a cross-sectional view of a sliding portion of the sliding support assembly shown in FIG. 7 according to at least one embodiment of the present disclosure.

FIG. 9 is a schematic diagram of the sliding portion of the sliding support assembly shown in FIG. 7 according to at least one embodiment of the present disclosure.

### DETAILED DESCRIPTION

Specific embodiments of the present disclosure will be described below. It should be noted that in the specific description of these embodiments, for the sake of brevity and conciseness, this specification may not describe all features of the actual embodiments in detail. It should be understood that in the actual implementation process of any embodiments, just as in the process of any engineering project or design project, a variety of specific decisions are often made to achieve specific goals of the developer and to meet system-related or business-related constraints, which may also vary from one embodiment to another. Furthermore, it should also be understood that although efforts made in such development processes may be complex and tedious, for those of ordinary skill in the art related to the content disclosed in the present disclosure, some design, manufacture, or production changes based on the technical content disclosed in the present disclosure are only common technical terms and should not be construed as insufficient content of the present disclosure.

Unless defined otherwise, technical terms or scientific terms used in the claims and specification should have the usual meanings understood by those of ordinary skill in the technical field to which the present disclosure belongs. The terms “first,” “second” and similar terms used in the description and claims of the patent application of the present disclosure do not denote any order, quantity, or importance, but are merely intended to distinguish between different constituents. The terms “one” or “a/an” and similar terms do not express a limitation of quantity, but rather that at least one is present. The terms “include” or “comprise,” and similar words indicate that an element or article preceding the terms “include” or “comprise” encompasses elements or articles and equivalent elements thereof listed after the terms “include” or “comprise,” and does not exclude other elements or articles. The terms “connect,” or “link” and similar words are not limited to physical or mechanical connections and are not limited to direct or indirect connections.

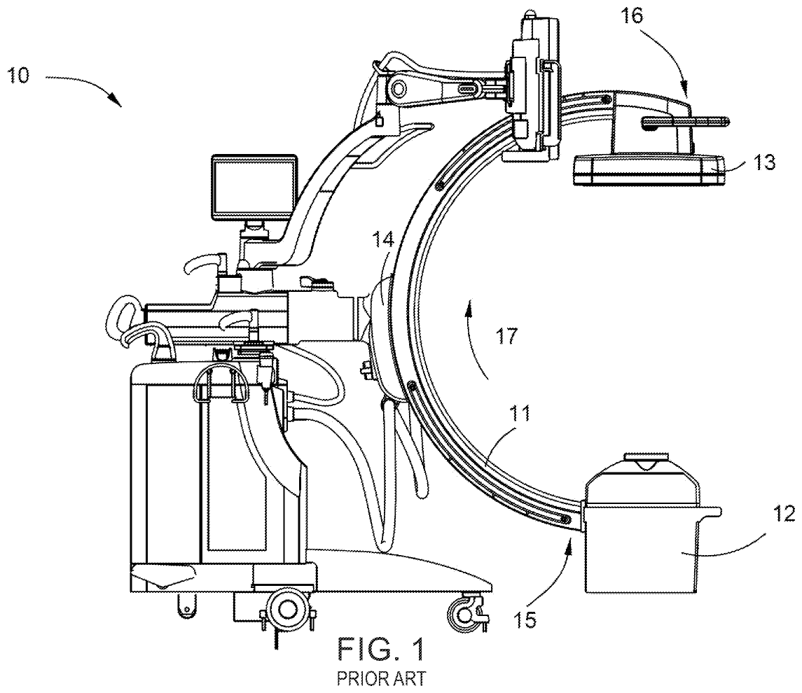

FIG. 1 illustrates a schematic diagram of a prior art C-arm imaging system 10 to illustrate limitations of the prior art design. As shown in FIG. 1, the C-arm imaging system 10 includes a C-arm 11 and a sliding support assembly 14, and a X-ray tube assembly 12 in which the X-ray source is located at one end portion 15 of the C-arm 11, and a detector assembly 13 is mounted at the other end portion 16 of the C-arm. In at least one embodiment, the X-ray tube assembly 12 is mounted on the side of the end portion 15, i.e., one side of the X-ray tube assembly 12 is connected to the end portion 15 of the C-arm 11. Typically, at least a portion of the X-ray tube assembly 12 (e.g., the X-ray tube as well as the transformer portion) is located on the outside of the C-arm 11, i.e., outside the outer circumference of the C-arm, and at least another portion of the X-ray tube assembly 12 (e.g., the collimator) is located within the inner circumference of the C-arm. The sliding support assembly 14 is mounted on the outer surface of the C-arm and is slidable relative to the C-arm, with the contact portion of the sliding support assembly 14 and the C-arm typically being provided on the transverse rotation axis of the C-arm, i.e., the transverse axis of symmetry of the C-arm.

In prior art C-arm imaging systems the angle of rotation of the C-arm cannot exceed 90 degrees when rotated in the direction 17 between one end of the sliding support assembly 14 near the X-ray tube assembly and the end portion 15 of the C-arm does not exceed 90 degrees. That is, when the C-arm is rotated in the clockwise direction, the X-ray tube assembly 12 cannot achieve 90° rotation when rotated in the direction 17 near the motion assembly 14 due to the blockage of the X-ray tube assembly. That is, the X-ray tube assembly cannot be rotated to a position where a line connecting the X-ray tube assembly with the center of the detector is in a horizontal direction, which causes that the C-arm has a limited rotation angle and cannot achieve positioning and shooting at certain specific positions or angles. Therefore, in order to be able to achieve the multi-angle positioning of the C-arm, the present disclosure proposes a C-arm imaging system that can achieve bidirectional 90-degree rotation when the sliding support assembly itself cannot rotate.

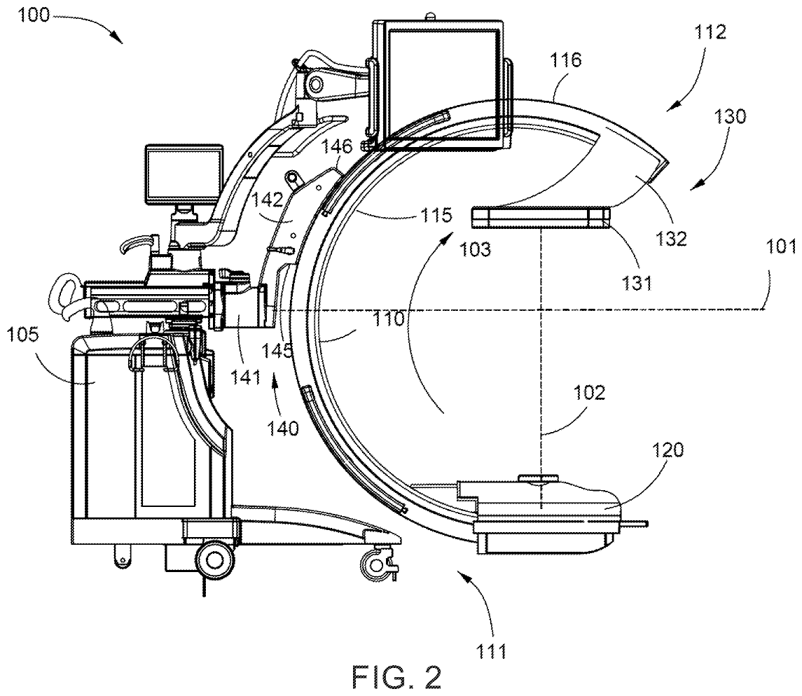

FIG. 2 shows a schematic diagram of a C-arm imaging system 100 at a first position according to at least one embodiment of the present disclosure. As shown in FIG. 2, the C-arm imaging system 100 includes a C-arm 110, an X-ray tube assembly 120, a detector assembly 130, and a sliding support assembly 140.

The C-arm imaging system 100 may utilize a plurality of imaging modalities (for example, fluoroscopy, computed tomography, tomosynthesis, X-radiography, and the like) to acquire two-dimensional (2D) and/or 3D image data. The C-arm imaging system 100 may be used for both diagnosis and interventional imaging. In addition, the C-arm imaging system 100 may be used for general purposes (for example, general radiology, orthopedics, or the like) and special purposes (for example, an image-guided surgery).

In at least one embodiment, the C-arm 110 includes an inner surface 115 and an outer surface 116 which are oppositely disposed. The inner surface and the outer surface terminate at the first end portion 111 and second end portion 112 which are opposite to each other, with the first end portion 111 and the second end portion 112 being oppositely disposed. In at least one embodiment, the C-arm 110 includes a uniform C shape. In at least one embodiment, the inner surface 115 is the inner circumference and the outer surface 116 is the outer circumference, but the present application is not limited thereto, and the C-arm 110 may also include any curved member.

The outer surface 116 of the C-arm 110 is not uniformly curved, and the outer side of the C-arm is provided with an arc-shaped recess that opens from the first end portion 111 toward the second end portion 112, which forms the sliding track of the C-arm. The outer surface in the present application refers to side walls and the bottom of the entire recess and the outer surface opposite to the inner surface.

The X-ray tube assembly 120 is mounted at the first end portion 111 of the C-arm and the detector assembly 130 is mounted at the second end portion 112 of the C-arm, and the detector assembly 130 and the X-ray tube assembly 120 are aligned, i.e., the center of the detector assembly is located at the center of the X-ray beam emitted by the X-ray tube assembly (or the X-ray source included therein). The C-arm allows the X-ray tube assembly and detector assembly to be mounted and positioned around the subject to be imaged. In at least one embodiment, the C-arm allows the X-ray tube assembly and detector assembly to be selectively positioned relative to the width and length of the patient or other subjects located within the interior space of the C-arm. In operation, a patient, for example, is placed on a table arranged in the space between the detector assembly and the X-ray tube assembly, and the C-arm is able to move or rotate to position the X-ray tube assembly and the detector assembly in a desired position relative to the patient to acquire a medical image of the patient.

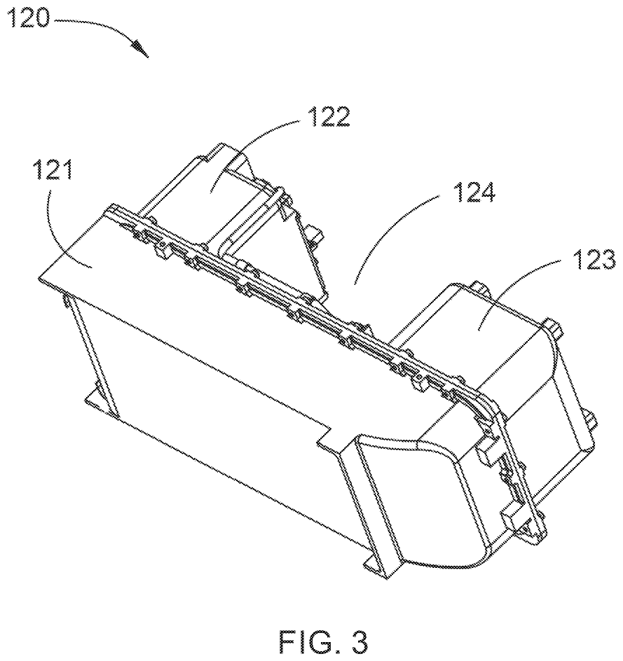

FIG. 3 illustrates a schematic diagram of the X-ray tube assembly of the C-arm imaging system shown in FIG. 2 according to at least one embodiment. For ease of display, the collimator is omitted in FIG. 3. However, a person of ordinary skill in the art should understand that a collimator is disposed within the gap 124 shown in FIG. 3. In at least one embodiment, the X-ray tube assembly 120 includes a body 121, a first extension portion 122, a second extension portion 123, and a collimator (not shown in the figure). The body 121 can be used to accommodate an X-ray tube, and the bottom of the body is disposed within the inner surface of the C-arm.

The first extension portion 122 is used to accommodate a first transformer. The second extension portion 123 is used to accommodate a second transformer, and a gap 124 is formed between the first extension portion 122 and the second extension portion 123. The collimator can be disposed within the gap 124, and an outlet end portion of the collimator is substantially aligned with an end portion of the first extension portion 122 and an end portion of the second extension portion 123.

The first extension portion 122 and the second extension portion 123 are disposed opposite to each other and above the body 121 (in an emission direction of X-rays). The body, the first extension portion, and the second extension portion may be integrally formed or connected together by welding, sticking, or the like. The first transformer may be a filament transformer, and the second transformer may be a high-voltage transformer.

In at least one embodiment, the first end portion of the first extension portion 122 is substantially aligned with the first end portion of the second extension portion 123, while the second end portion of the first extension portion and the second end portion of the second extension portion are respectively connected to the body. The body, the first extension portion and the second extension portion are substantially formed in a concave shape. By recessing the collimator, the height of the X-ray tube assembly may be reduced in comparison to conventional configurations, thereby increasing the distance and space between a detector surface of the detector assembly and an outlet of the X-ray tube assembly.

The X-ray tube assembly 120 is mounted on the side edge of the first end portion 111 of the C-arm, i.e., the side edge of the X-ray tube assembly is in contact with the side edge of the end portion of the C-arm to further increase the size of the space between the detector and the X-ray tube.

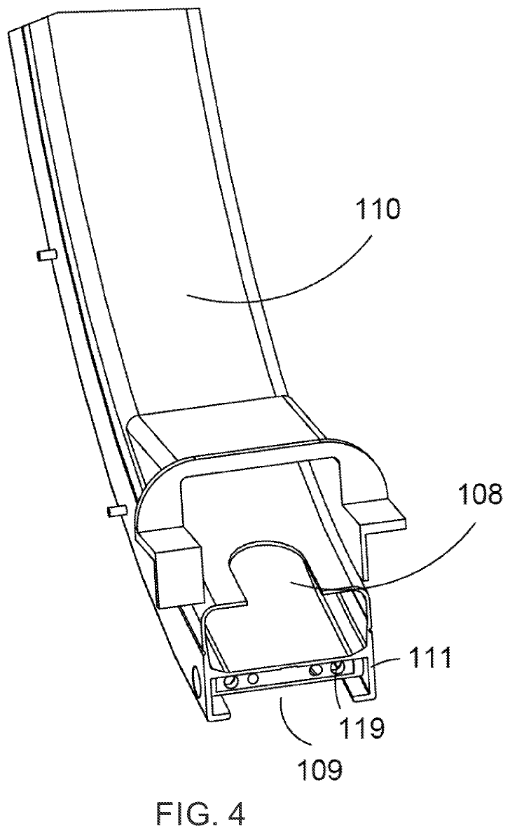

FIG. 4 illustrates a schematic diagram of the C-arm shown in FIG. 2 according to at least one embodiment of the present disclosure. As shown in FIG. 4, the first end portion 111 of the C-arm 111 is machined into a flat surface, and a plurality of positioning holes 119 are provided on the side edge near the bottom of the sliding track 109 (or the recess on the C surface), and a plurality of positioning pins are provided on the side edge of the X-ray tube assembly. Alignment of the X-ray tube assembly and the C-arm can be achieved by aligning the positioning pins on the X-ray tube assembly with the positioning holes on the side edge of the C-arm, and then the X-ray tube assembly is fixed or mounted on the C-arm by screws or other means to achieve side mounting of the X-ray tube assembly. In at least one embodiment, two positioning holes are provided at the end portion of the C-arm, and two positioning pins are provided on the side edge of the X-ray tube assembly accordingly. By mounting the X-ray tube at the end portion of the C-arm, i.e., side mounting, the convenience of fixing and positioning the X-ray tube is increased on the one hand, and in addition, the bottom of the tube is in contact with the air, which is more conducive to the heat dissipation of the X-ray tube.

In at least one embodiment, the inner surface on the C-arm near the end portion further includes a wiring slot 108 that can be used to accommodate various cables, etc., for connecting components such as the X-ray tube assembly. In at least one embodiment, the C-arm is further mounted with an extension housing which extends horizontally from the top of the X-ray tube assembly to the C-arm, i.e. the top of the extension housing is substantially flush with the top plane of the X-ray tube assembly, and the extension housing is a reinforcement, which can increase the rigidity of the C-arm and improve the bending resistance on the one hand, and play the role of sealing and waterproofing on the other hand.

Of course, a person of ordinary skills in the art should understand that it is also possible to mount the X-ray tube assembly 120 on the inside of the first end portion of the C-arm so that the outer surface of the section of the C-arm corresponding to the section where the X-ray tube assembly is mounted can also form a sliding track, i.e., the sliding support assembly can slide to the outer surface corresponding to the mounting position of the X-ray tube assembly.

Please refer back to FIG. 2, during an imaging operation, a part of the patient's body placed in a space (for example, a gap) formed between the X-ray tube assembly 120 and the detector assembly 130 may be irradiated using radiation from an X-ray source. For example, X-ray radiation generated by the X-ray source may penetrate the irradiated part of the patient's body and propagate to the detector assembly 130 where the radiation is captured. The part of the patient's body placed between the X-ray tube assembly 120 and the detector assembly 130 is penetrated, so that an image of the patient's body is captured and relayed to the electronic controller of the imaging system 100 (for example, via an electrical connection line such as a conductive cable).

The sliding support assembly 140 provides stable and balanced support for the C-arm. For example, the sliding support assembly 140 allows the C-arm to be suspended for imaging of the patient or subject. The sliding support assembly 140 also enables the C-arm to rotate about an axis of rotation (e.g., manually or using an electric motor). For example, the C-arm can rotate along the transverse rotation axis 101.

The sliding support assembly 140 is mounted on the outer surface of the C-arm, and the C-arm is capable of moving relative to the sliding support assembly 140. The sliding support assembly 140 includes a sliding member that fits into a sliding track or recess of the C-arm, and at least a portion of the sliding member is provided within the sliding track and can slide on the sliding track, thereby enabling the C-arm to slide relative to the sliding support assembly.

In at least one embodiment, the C-arm imaging system further includes a support structure or support base 105. The support base 105 supports the C-arm 110 and maintains the C-arm 110 at a suspended position. The lower part of the support base 105 includes wheels or casters used for providing mobility to the system 100.

The support base 105 may include an electronic controller (for example, a control and computing unit), and the electronic controller processes instructions or commands sent from a user input device during operation of the imaging system 100. The support base 105 may further include an internal power supply (not shown), and the internal power supply provides power to operate the imaging system 100. Alternatively, the support base 105 may be connected to an external power supply to facilitate power supply to the imaging system 100. A plurality of connection lines (for example, power cables such as conductive cables) may be provided to transfer power, instructions, and/or data between the X-ray tube assembly 120, the detector assembly 130, and the control and computing unit. The plurality of connection lines may transfer power from a power supply (for example, an internal source and/or an external source) to the X-ray tube assembly 120 and the detector assembly 130. For ease of display, the power cable structure is omitted in FIG. 2. The power cable in the C-arm imaging system can be connected from the support base to the C-arm, and can also be connected from the support base to devices such as a display or display screen, etc.

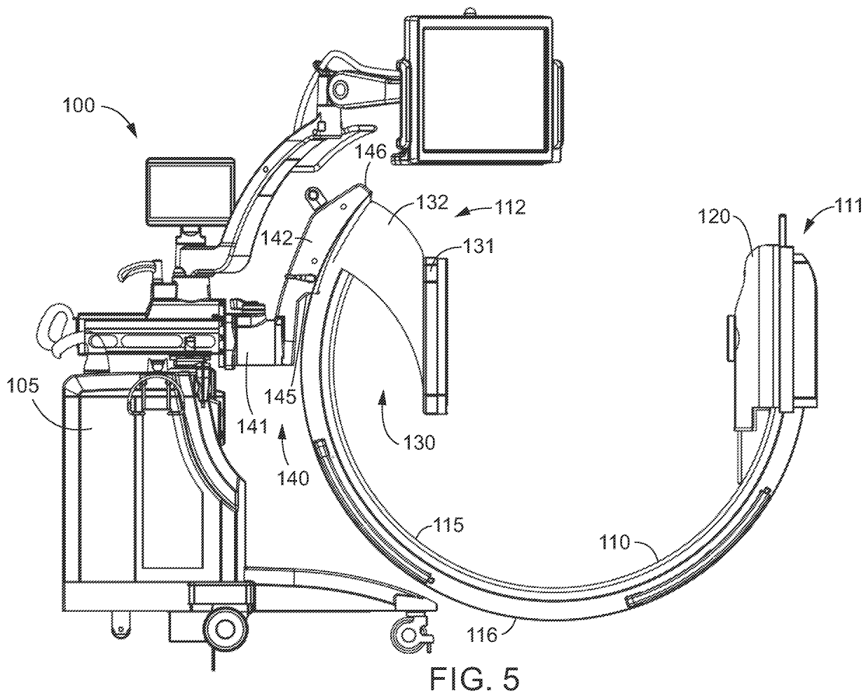

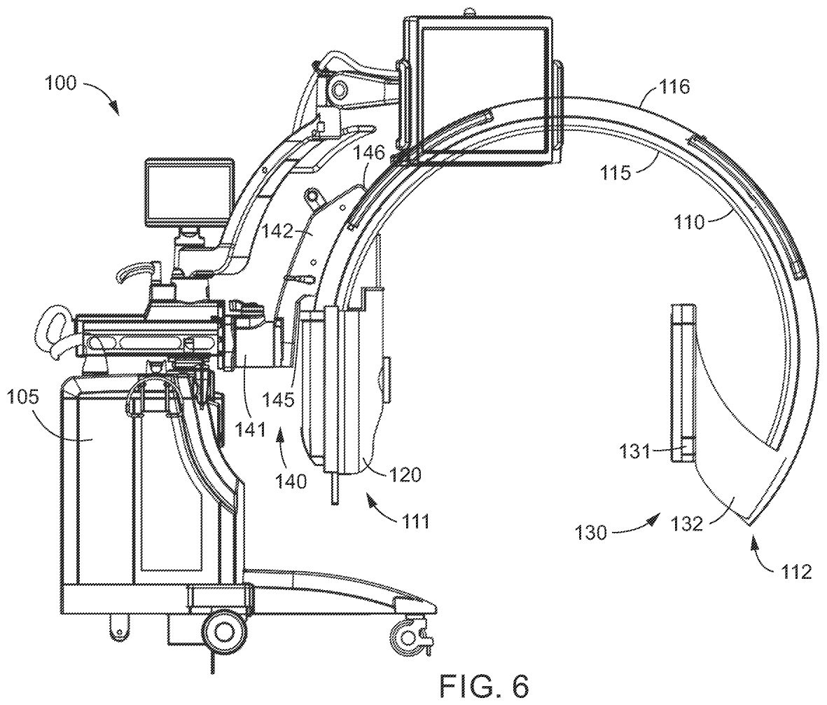

FIG. 5 illustrates a schematic diagram of the C-arm imaging system in a second position according to at least one embodiment of the present disclosure and FIG. 6 illustrates a schematic diagram of the C-arm imaging system in a third position according to at least one embodiment of the present disclosure.

As shown in FIG. 2 and FIGS. 5-6, for ease of description, in the present application, the first position (or initial position) is defined as a position where the line connecting the center of the detector assembly and the center of the X-ray tube assembly is in a vertical direction as shown in FIG. 2, i.e., the detector assembly is located directly above the X-ray tube assembly. The second position is as shown in FIG. 5, where a line connecting the center of the detector assembly and the center of the X-ray tube assembly is in a horizontal direction, and the detector assembly slides to the opposite side of the sliding support assembly, i.e., the detector assembly is located directly to the left of the X-ray tube assembly. The third position is shown in FIG. 6, where the line connecting the center of the detector assembly and the center of the X-ray tube assembly is in a horizontal direction, and the X-ray tube assembly slides to the opposite side of the sliding support assembly, i.e., the detector assembly is located directly to the right of the X-ray tube assembly.

In at least one embodiment, the second position can be reached by sliding the C-arm in a counterclockwise direction from the first position, and the third position can be reached by sliding the C-arm in a clockwise direction from the first position. In some descriptions, the second position is an “upside down C” position of the C-arm, where most of the C-arm is below the transverse rotation axis, while the third position is an “upright C” position of the C-arm, where most of the C-arm is above the transverse rotation axis.

In at least one embodiment, the C-arm of the present application rotates from the first position to the first end portion or the second end portion by an angle of rotation of the C-arm not less than 90 degrees. In at least one embodiment, the angle at which the C-arm rotates from the first position to the second position, or the third position is not less than 90 degrees, i.e., the angle at which the C-arm slides from the first position in the clockwise or counterclockwise direction is not less than 90 degrees.

In at least one embodiment, the sliding support assembly 140 further includes a pivoting portion 141 connected to the support base 105, and a sliding portion 142 connected to the C-arm. The axis of the pivoting portion 141 is on the transverse rotation axis 101 of the C-arm, and the sliding portion of the sliding support assembly has a predetermined distance from the transverse rotation axis. In at least one embodiment, the pivoting portion 141 is coupled to the horizontal extension arm 106 of the support base 105, and the sliding support assembly is capable of pivoting or rotating about the horizontal extension arm 106 on a pivot axis, which in turn drives the C-arm to pivot or rotate about the horizontal extension arm 106 on the pivot axis. In at least one embodiment, the axis of the horizontal extension arm and the axis of the pivoting portion 141 may be the transverse rotation axis 101. In at least one embodiment, the C-arm is capable of rotating 360 degrees in a circumferential direction around the transverse rotation axis 101.

In at least one embodiment, the position of the sliding portion of the sliding support assembly relative to the pivoting portion is fixed. In at least one embodiment, the sliding support assembly itself of the present application is not rotatable, and more specifically, the sliding portion is above the pivoting portion, i.e., the sliding portion cannot be adjusted below the pivoting portion, i.e., the position of the sliding portion relative to the support base or relative to the pivoting portion is fixed in the first position, the second position, the third position, or any other angle or position.

In at least one embodiment, the sliding portion 142 of the sliding support assembly 140 includes a first end 145 proximate to the X-ray tube assembly (or the first end portion 111 of the C-arm) and a second end 146 proximate to the detector assembly (or the second end portion 112 of the C-arm), and the angle between the second end 146 of the sliding portion and the second end portion 112 of the C-arm is not less than 90 degrees. In at least one embodiment, the angle of rotation between the first end 145 of the sliding portion and the first end portion 111 of the C-arm is also not less than 90 degrees.

In at least one embodiment, the angle between the second end 146 of the sliding portion and the second end portion 112 of the C-arm is the angle between the line connecting the second end of the sliding portion and the center of the circumference in which the C-arm is located and the line connecting the second end portion 112 and the center, and similarly, the angle between the first end 145 of the sliding portion and the first end portion 111 of the C-arm is the angle between the line connecting the second end of the sliding portion and the center and the line connecting the first end portion 111 and the center.

In at least one embodiment, the C-arm is fitted with a position-limiting device on the side edges near the first end portion and the second end portion for limiting the extent to which the C-arm rotates relative to the sliding support assembly. In at least one embodiment, the position-limiting device may also be mounted on the sliding portion 146, and more specifically, on the first end and second end of the sliding portion 146.

In at least one embodiment, the C-arm 110 includes a first segment and a second segment extending in the circumferential direction of the C-arm, the end portion of the second end being the second end portion 112 of the C-arm, and the arc of the second segment being substantially the arc between the connection portion of the sliding support assembly to the C-arm relative to the transverse rotation axis.

In at least one embodiment, the C-arm near the detector assembly is to exceed the longitudinal axis direction 102, wherein the longitudinal axis direction 102 is the direction in which the line connecting the center of the detector to the center of the X-ray tube is located, and the second end portion of the C-arm is substantially flush with the outermost side of the X-ray tube assembly, wherein the outermost side of the X-ray tube assembly is the other end opposite to the end where the C-arm is mounted, i.e., the first end portion and second end portion of the C-arm are not symmetrical with respect to the transverse rotation axis 101.

In at least one embodiment, as the X-ray tube assembly is mounted at the end portion of the C-arm, in order to ensure that the X-ray tube assembly can slide to the “upright C” position, i.e., the X-ray tube assembly is located directly to the left of the detector assembly, the sliding portion of the sliding support assembly is raised by a predetermined distance, i.e., the sliding portion has a predetermined distance from the transverse rotation axis so that the angle between the first end 145 of the sliding portion and the first end portion of the C-arm is 90 degrees. Due to the elevation of the sliding portion, the arc length of the original C-arm cannot satisfy that the angle between the second end of the sliding portion and the second end portion is also 90 degrees. Therefore, the present disclosure proposes to extend the C-arm in the circumferential direction on the basis of the first segment (the circumference of the original C-arm), and the arc length of the extended second segment is substantially the arc length between the second end 146 of the sliding support assembly and the connection point between the C-arm and the transverse rotation axis, so that the arc of the sliding track of the C-arm is not less than 180 degrees. In at least one embodiment, the C-arm has an arc that is greater than 180 degrees, which is substantially equal to or slightly greater than 180 degrees added with the angle between the first end and the second end of the sliding portion, i.e., the opening angle of the sliding portion. That is, the arc length of the C-arm is larger than a semicircle.

In at least one embodiment, as the C-arm is extended in the direction of the second end portion, in order to allow the detector assembly and the X-ray tube assembly to remain aligned, in the present application, the connector is disposed between the detector and the second end portion to be positioned obliquely. In at least one embodiment, the detector assembly 130 includes a detector 131 and a connector 132 for connecting between the detector and the second end portion 112 of the C-arm, with the connector 132 positioned obliquely inward and the end of the connector 132 that is connected to the detector is arranged closer to the support base than the other end. In at least one embodiment, in the first position, the connector is positioned obliquely from the upper right toward the lower left.

In at least one embodiment, the obliquity of the connector is determined on the conditions of aligning the detector to the center of the X-ray tube assembly and forming an angle not less than 90 degrees between the sliding portion and the second end portion, i.e., by arranging the connector, it is necessary to satisfy, on the one hand, an angle not less than 90 degrees between the sliding portion (or the second segment 146 of the sliding portion) and the second end portion of the C-arm, and on the other hand, it is also necessary to satisfy the alignment of the center of the detector to the center of the X-ray tube assembly (i.e., the opening of the collimator or the central ray of the X-ray beam).

Only one form of application or shape of the connector is shown in the figure, and it should be understood by those skilled in the art that the connector can also be arranged to be any size or shape, as long as the two conditions mentioned above can be met.

In at least one embodiment, the connector can be made of plastic, or resin or metal, for example. The connector and the detector housing can be molded in one piece, or the connector can be fixed thereon in any form by welding, gluing, etc.

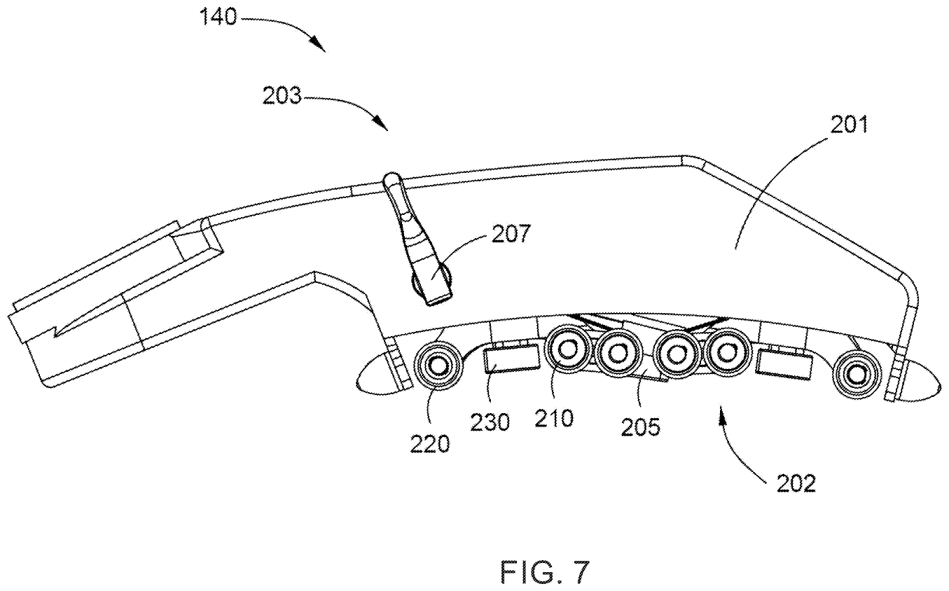

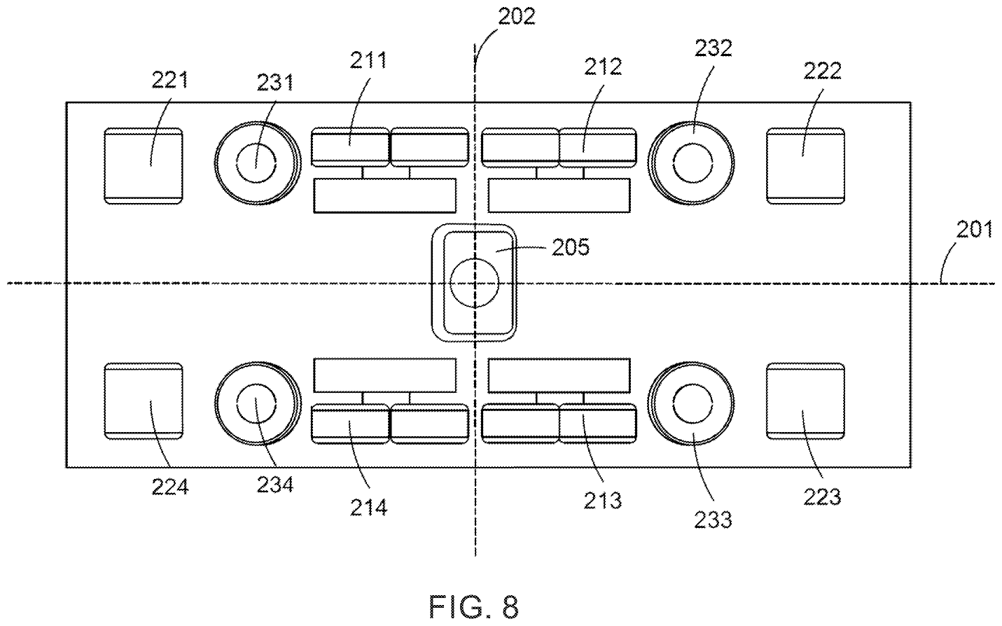

FIG. 7 illustrates a schematic diagram of a sliding support assembly of a C-arm imaging system according to at least one embodiment of the present disclosure and FIG. 8 illustrates a cross-sectional view of a sliding portion of the sliding support assembly shown in FIG. 7 according to at least one embodiment of the present disclosure.

As shown in FIGS. 6-7, the sliding support assembly 140 includes a body portion 201 and a roller portion 202, and the roller portion 202 is provided within the sliding track of the C-arm, and the roller portion 202 is capable of rolling along the direction of the sliding track. In at least one embodiment, the sliding portion further includes a clutch and brake assembly 203 configured to apply both braking force and clutch force to the C-arm. The clutch and brake assembly 203 includes a braking member 205 and a lever 207, and the actuation of the lever 207 regulates the position of the brake liner block (not shown in the figure) relative to the roll 205. For example, the lever 207 can be rotated, so that the brake liner block contacts the roll 205 to cause the roll 205 to apply braking force to the C-arm 16.

The sliding portion 142 to which the sliding support assembly 140 and the C-arm 100 are attached includes four sets of symmetrical rolling wheels 210, four sets of corner wheels 220 located at the four corners of the sliding support assembly, and four sets of side wheels 230 located between the rolling wheels 210 and the corner wheels 220, with the side wheels 230 rolling along the side edges of the track of the C-arm and the rolling wheels 210 and the corner wheels 220 rolling along the bottom edge of the track of the C-arm. In at least one embodiment, the rolling wheels 210 and the corner wheels 220 are capable of rolling along the track direction 201 of the C-arm.

In at least one embodiment, as shown in FIG. 8, the sliding portion includes a first set of rolling wheels 211, a second set of rolling wheels 212, a third set of rolling wheels 213, and a fourth set of rolling wheels 214, and these four sets of rolling wheels are symmetrically arranged at the center position of the sliding portion, and arranged around the roll 205. In at least one embodiment, each set of rolling wheels includes two rolling wheels, and the two rolling wheels are arranged along a reference direction 202 perpendicular to the track direction 201.

In at least one embodiment, the sliding portion includes a first set of corner wheels 221, a second set of corner wheels 222, a third set of corner wheels 223, and a fourth set of corner wheels 224, and these four sets of corner wheels are symmetrically arranged on the four corners of the sliding section. In at least one embodiment, each set of corner wheels includes one wheel that has a larger width in the reference direction 202. As space conditions permit, a larger width of the wheel can increase the sliding stability of the C-arm, in addition to extending the service life of the wheel.

In at least one embodiment, the sliding portion includes a first set of side wheels 231, a second set of side wheels 232, a third set of side wheels 233, and a fourth set of side wheels 234, and these four sets of side wheels are symmetrically arranged between the corresponding rolling wheels and corner wheels. In at least one embodiment, each set of side wheels includes one wheel.

Although each set of corner wheels is shown in FIG. 8 to include one wider wheel, it should be understood by those skilled in the art that two wheels may be provided along the reference direction 202.

In at least one embodiment, the sliding portion further includes a connection unit connected to the rolling wheel for securing the rolling wheel. Of course, although many non-essential units or members are omitted from the drawings, it should be understood by those skilled in the art that each set of corner wheels and side wheels also has corresponding connection units, as well as other units or members for securing and mounting.

Compared with each set of corner wheels including two wheels arranged along the track direction 201, each set of corner wheels in the present application includes only one wider wheel. That is to say, along the track direction 201, each end saves a wheel space, making the opening angle of the sliding portion smaller. That is, the area of the contact portion with the C-arm is also reduced, and also the full arc length of the C-arm is controlled as much as possible, which can save cost on the one hand, and reduce the weight of the C-arm on the other hand, thereby making the entire system more lightweight.

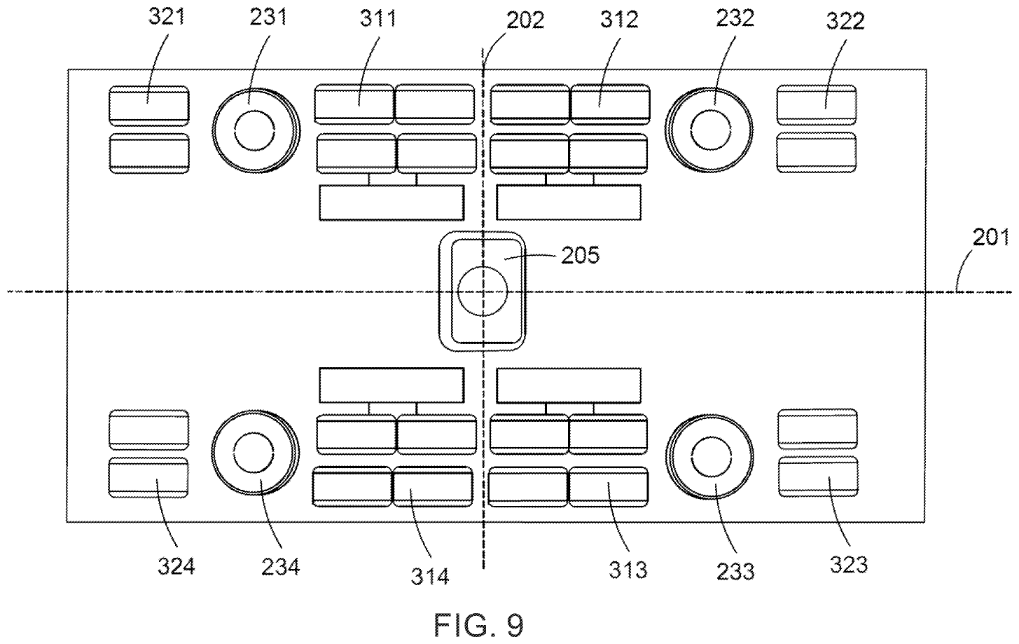

FIG. 9 illustrates a schematic diagram of the sliding portion of the sliding support assembly shown in FIG. 7 according to at least one embodiment of the present disclosure. As shown in FIG. 9, unlike the sliding portion shown in FIG. 8, the rolling wheels and corner wheels in the sliding portion shown in FIG. 9 near the side wall of the C-arm are provided with an additional wheel along the reference direction 202, i.e., each set of rolling wheels 311, 312, 313, 314 includes two rows and two columns of a total of four rolling wheels, with the two rows of rolling wheels arranged along the track direction 201 and the two columns of rolling wheels arranged along the reference direction 202. Each set of corner wheels includes two columns of corner wheels, and the two columns of corner wheels are arranged along the reference direction 202. By adding an extra row of wheels in the reference direction, the bearing capacity of the rotation assembly can be further increased to improve the rotation stability and reliability of the C-arm on the one hand, and the service life of the wheels can be extended on the other hand.

For the C-arm imaging system in at least one embodiment of the present disclosure, by extending the C-arm to a certain degree in the circumferential direction, the sliding distance of the C-arm is not less than 180 degrees, and furthermore, by raising the sliding portion of the sliding support assembly, the C-arm can achieve the “upright C” and “upside-down C” positions even though the sliding support assembly itself cannot rotate, which on the one hand can reduce the height of the X-ray tube assembly, thereby increasing the distance from the outlet of the X-ray tube assembly to the detector surface of the detector assembly, i.e., increasing the overall size of the patient accommodation space, which allows the C-arm to accommodate patients with larger sizes for imaging and/or increases the ease of use of the C-arm. On the other hand, such design enables the C-arm to be rotated 180 degrees in various directions relative to the sliding support assembly, and achieves the angle at which the detector assembly and the X-ray tube assembly are parallel to the rotation axis and the X-ray tube assembly is opposite to the sliding support assembly, thereby achieving wider and more flexible angle adjustments.

Secondly, by improving the sliding portion of the sliding support assembly, the sliding portion has a smaller opening angle, thereby achieving a more compact structure design, on the one hand, more aesthetic, and on the other hand, the amplitude of the extension of the C-arm can also be reduced, so that the entire C-arm imaging system is also more aesthetic and compact.

In at least one embodiment of the present disclosure have provided a C-arm imaging system, the system including a C-arm and a sliding support assembly. The C-arm includes a first end portion and a second end portion which are oppositely disposed. The first end portion is used to connect to the X-ray tube assembly, while the second end portion is used to connect to the detector assembly, and the X-ray tube assembly and the detector assembly are aligned. The sliding support assembly is connected to the C-arm, and the C-arm can slide relative to the sliding support assembly, wherein the C-arm slides at an angle not less than 90 degrees relative to the sliding support assembly from a first position to the first end portion or the second end portion, the first position being a position where the line connecting the center of the detector assembly and the center of the X-ray tube assembly is in a vertical direction.

In at least one embodiment the C-arm imaging system further includes a support base, and the sliding support assembly includes a pivoting portion connected to the support base and a sliding portion connected to the C-arm, the axis of the pivoting portion being on a transverse rotation axis of the C-arm, and the sliding portion of the sliding support assembly having a predetermined distance from the transverse rotation axis.

In at least one embodiment, the position of the sliding portion of the sliding support assembly relative to the pivoting portion is fixed.

In at least one embodiment, the sliding portion is located above the pivoting portion.

In at least one embodiment, the sliding portion includes a first end proximate to the X-ray tube assembly and a second end proximate to the detector assembly, and the angle between the second end of the sliding portion and the second end portion of the C-arm is not less than 90 degrees.

In at least one embodiment, the C-arm includes a first segment and a second segment extending in a circumferential direction along the first segment, one end of the second segment being the second portion, and the arc of the second segment is substantially the arc between the connection portion of the sliding support assembly to the C-arm relative to the transverse rotation axis.

In at least one embodiment, the detector assembly includes a detector, and a connector for connecting between the detector and a second end portion of the C-arm, the connector being positioned obliquely inward.

In at least one embodiment, the sliding portion connecting the sliding support assembly and the C-arm includes four sets of symmetrical rolling wheels, four sets of corner wheels located at the four corners of the sliding support assembly, and four sets of side wheels located between the rolling wheels and corner wheels, the side wheels rolling along the side edges of the track of the C-arm, and the rolling wheels and the corner wheels rolling along the bottom edge of the track of the C-arm.

In at least one embodiment, each set of the rolling wheels includes two or four wheels, and each set of corner wheels includes one or two wheels, and when each set of corner wheels includes two wheels, the two wheels are arranged in a direction perpendicular to the sliding direction of the corner wheels.

In at least one embodiment, the X-ray tube assembly is mounted at the end portion of the C-arm, and the X-ray tube assembly includes an X-ray tube unit disposed in a concave shape and a collimator unit mounted in a recess, the top of the X-ray tube being substantially flush with the top of the collimator.

Illustrative embodiments of the present disclosure have further provided a C-arm imaging system, the imaging system including a C-arm, an X-ray tube assembly, a detector assembly, and a sliding support assembly. The C-arm has a recess provided on an outer surface thereof, and the recess forms a sliding track of the C-arm. The X-ray tube assembly is mounted at the first end portion of the C-arm. The detector assembly is mounted at the second end portion of the C-arm. The sliding support assembly is connected to the outer surface of the C-arm, and the sliding support assembly includes a sliding portion, at least a portion of the sliding portion being provided within the sliding track, wherein the sliding portion is at an angle not less than 90 degrees from a first position to the first end portion or second end portion, and the first position is a position where the line connecting the center of the detector assembly and the center of the X-ray tube assembly is in a vertical direction.

In at least one embodiment, the C-arm imaging system further includes a support base, and the sliding support assembly includes a pivoting portion connected to the support base, the axis of the pivoting portion being on a transverse rotation axis of the C-arm, and the sliding portion having a predetermined distance from the transverse rotation axis.

In at least one embodiment, the position of the sliding portion relative to the pivoting portion is fixed.

In at least one embodiment, the sliding portion is located above the pivoting portion.

In at least one embodiment, the C-arm includes a first segment and a second segment extending in a circumferential direction along the first segment, one end of the second segment being the second port, and the arc of the second segment is substantially the arc between the connection portion of the sliding portion to the C-arm relative to the transverse rotation axis.

In at least one embodiment, the detector assembly includes a detector, and a connector for connecting between the detector and a second end portion of the C-arm, the connector being positioned obliquely inward.

In at least one embodiment, the sliding portion includes four sets of symmetrical rolling wheels, four sets of corner wheels located at the four corners of the sliding portion, and four sets of side wheels located between the rolling wheels and corner wheels, the side wheels rolling along the side edges of the sliding track, and the rolling wheels and the corner wheels rolling along the bottom edge of the sliding track.

In at least one embodiment, each set of the rolling wheels includes two or four wheels, and each set of corner wheels includes one or two wheels, and when each set of corner wheels includes two wheels, the two wheels are arranged in a direction perpendicular to the sliding direction of the corner wheels.

In at least one embodiment, the X-ray tube assembly is mounted at the end portion of the C-arm, and the X-ray tube assembly includes an X-ray tube disposed in a concave shape and a collimator unit mounted in a recess, the top of the X-ray tube being substantially flush with the top of the collimator.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

1. A C-arm imaging system, comprising:
a C-arm which comprises a first end portion and a second end portion which are oppositely disposed, the first end portion for connecting to an X-ray tube assembly, the second end portion for connecting to a detector assembly, and the X-ray tube assembly and the detector assembly being aligned; and
a sliding support assembly which is connected to the C-arm, and the C-arm is capable of sliding relative to the sliding support assembly;
wherein the C-arm slides at an angle not less than 90 degrees relative to the sliding support assembly from a first position to the first end portion or the second end portion, the first position being a position where the line connecting the center of the detector assembly and the center of the X-ray tube assembly is in a vertical direction.

2. The C-arm imaging system according to claim 1, wherein the C-arm imaging system further comprises a support base, and the sliding support assembly comprises a pivoting portion connected to the support base and a sliding portion connected to the C-arm, the axis of the pivoting portion being on a transverse rotation axis of the C-arm, and the sliding portion of the sliding support assembly having a predetermined distance from the transverse rotation axis.

3. The C-arm imaging system according to claim 2, wherein the position of the sliding portion of the sliding support assembly relative to the pivoting portion is fixed.

4. The C-arm imaging system according to claim 2, wherein the sliding portion is located above the pivoting portion.

5. The C-arm imaging system according to claim 2, wherein the sliding portion comprises a first end proximate to the X-ray tube assembly and a second end proximate to the detector assembly, and the angle of rotation between the second end of the sliding portion and the second end portion of the C-arm is not less than 90 degrees.

6. The C-arm imaging system according to claim 1, wherein the C-arm comprises a first segment and a second segment extending along a circumferential direction of the first segment, one end of the second segment being the second portion, and the arc of the second segment is substantially the arc between the connection portion of the sliding support assembly to the C-arm relative to the transverse rotation axis.

7. The C-arm imaging system according to claim 6, wherein the detector assembly comprises a detector, and a connector for connecting between the detector and the second end portion of the C-arm, the connector being positioned obliquely inward.

8. The C-arm imaging system according to claim 1, wherein the sliding portion of the sliding support assembly connected to the C-arm comprises four sets of symmetrical rolling wheels, four sets of corner wheels located at the four corners of the sliding support assembly, and four sets of side wheels located between the rolling wheels and the corner wheels, the side wheels rolling along the side edges of the track of the C-arm, and the rolling wheels and the corner wheels rolling along the bottom edge of the track of the C-arm.

9. The C-arm imaging system according to claim 8, wherein each set of the rolling wheels comprises two or four wheels, and each set of corner wheels comprises one or two wheels, and when each set of corner wheels comprises two wheels, the two wheels are arranged in a direction perpendicular to the sliding direction of the corner wheels.

10. The C-arm imaging system according to claim 1, wherein the X-ray tube assembly is mounted at the end portion of the C-arm, the X-ray tube assembly comprising an X-ray tube disposed in a concave shape and a collimator unit mounted in a recess, the top of the X-ray tube being substantially flush with the top of the collimator.

11. A C-arm imaging system, comprising:
a C-arm, having a recess provided on the outer surface thereof, the recess forming a sliding track for the C-arm;
an X-ray tube assembly, mounted at a first end portion of the C-arm;
a detector assembly, mounted at a second end portion of the C-arm; and
a sliding support assembly, connected to an outer surface of the C-arm, and the sliding support assembly comprising a sliding portion, at least a portion of the sliding portion being provided within the sliding track;
wherein the c-arm can rotate along the sliding portion not less than 90 degrees from a first position to the first end portion or second end portion, the first position being a position where the line connecting the center of the detector assembly and the center of the X-ray tube assembly is in a vertical direction.

12. A C-arm imaging system, comprising:
a C-arm, having a recess provided on the outer surface thereof, the recess forming a sliding track for the C-arm;
an X-ray tube assembly, mounted at a first end portion of the C-arm;
a detector assembly, mounted at a second end portion of the C-arm; and
a sliding support assembly, connected to an outer surface of the C-arm, and the sliding support assembly comprising a sliding portion, at least a portion of the sliding portion being provided within the sliding track;
wherein the C-arm can rotate along the sliding portion is at an angle not less than 90 degrees from a first position to the first end portion or second end portion, the first position being a position where the line connecting the center of the detector assembly and the center of the X-ray tube assembly is in a horizontal direction.
